(19) United States
(12) Patent Application Publication
(10) Pub. No.: US 2012/0065320 A1
(43) Pub. Date: Mar. 15, 2012

(54) FLUORINE-CONTAINING 1,6-DIENE ETHER COMPOUND AND FLUORINE-CONTAINING POLYMER

(75) Inventors: Toshio Kubota, Ibaraki (JP); Ko-ichi Yanai, Ibaraki (JP); Takehiro Nagasawa, Chiba (JP); Akira Hirooka, Chiba (JP); Makoto Hirooka, Gunma (JP); Eiko Hirooka, Gunma (JP)

(73) Assignees: NISSAN CHEMICAL INDUSTRIES, LTD., Tokyo (JP); IBARAKI UNIVERSITY, Mito-shi, Ibaraki (JP)

(21) Appl. No.: 13/321,311

(22) PCT Filed: May 18, 2010
(86) PCT No.: PCT/JP2010/058327

§ 371 Date: Dec. 1, 2011

(30) Foreign Application Priority Data
May 19, 2009 (JP) 2009-120528

(51) Int. Cl. C08F 16/32; C07C 43/192; C09D 129/10

(52) U.S. Cl. 524/544; 526/247; 568/667; 568/664

(57) ABSTRACT

A fluorine-containing polymer obtained using a 1,6-diene ether compound represented by formula [1] is a high-performance polymer exhibiting a low refractive index, high glass transition point, a high degree of transparency, and solubility in solvents. There are many potential uses, such as use as a coating material or bulk material. For example, said polymer could be effectively used in high-tech fields such as: optical materials such as low-reflection films and optical waveguide cladding; semiconductor materials such as pellicles and resists in semiconductor lithography; and protective film materials, insulating film materials, and water-repellent materials.

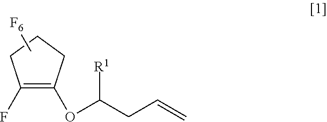

(In the formula, R¹ represents a C₆₋₁₈ aryl group or a C₁₋₁₂ alkyl group, which may be substituted.)

### TECHNICAL FIELD

This invention relates to a fluorine-containing 1,6-diene ether compound, and a fluorine-containing polymer obtained by use thereof and also to a method for preparing same.

### BACKGROUND ART

Although polytetrafluoroethylene (PTFE), which is typical of fluorine-based polymers, exhibits high heat and chemical resistances, it is opaque because of crystallinity thereof.

Cytop (registered trademark) and Teflon (registered trademark) AF are polymers that are amorphous and solvent-soluble, and are utilized as a low-reflection film. Nevertheless, limitation is placed on their use because of their low glass transition point.

On the other hand, octafluorocylopentene (OFCP) is a cycloolefin that has been industrially prepared. However, its use as a monomer is very rare since it is poor in polymerizability.

The present applicant has already reported that there is obtained a fluorine-containing polymer having a high glass transition point by polymerizing a 1,6-diene ether compound obtained by reaction between OFCP and a homoallyl alcohol (see Patent Document 1).

However, there have never been obtained materials that satisfy all characteristics including a refractive index, a heat resistance and a glass transition point.

### PRIOR-ART DOCUMENT

### Patent Document

Patent Document 1: JP-A 2007-314586

### SUMMARY OF THE INVENTION

### Problems to be Solved by the Invention

The invention has been made under such circumstances as set out above, and it is an object to provide a fluorine-containing 1,6-diene ether compound capable of yielding a fluorine-containing polymer that is low in refractive index, high in glass transition point, high in transparency and soluble in solvent, and also of a fluorine-containing polymer obtained therefrom and a method for preparing same.

### Means for Solving the Problems

We have made intensive studies so as to achieve the above object and, as a result, found that when using a specific type of fluorine-containing 1,6-diene ether compound as a starting monomer, a high-functional fluorine-containing polymer exhibiting a low refractive index, high glass transition point, high transparency and solvent solubility is obtained, thereby arriving at completion of the invention.

More particularly, the invention provides:

1. A 1,6-diene ether compound, characterized by being represented by the formula [1]:

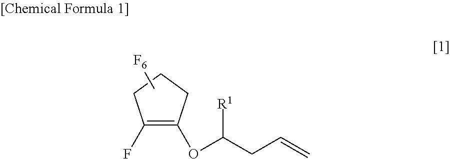

(wherein R¹ represents an alkyl group having 1 to 12 carbon atoms or an aryl group having 6 to 18 carbon atoms, which may be substituted);
2. The 1,6-diene ether compound of 1, wherein R¹ is an alkyl group having 1 to 12 carbon atoms, which may be substituted;
3. The 1,6-diene ether compound of 2, wherein R¹ is an alkyl group having 1 to 12 carbon atoms or a fluoroalkyl group having 1 to 12 carbon atoms;
4. A fluorine-containing polymer, characterized by being obtained by polymerizing a 1,6-diene ether compound of 2 or 3, or polymerizing a 1,6-diene ether compound of 2 or 3 and a 1,6-diene ether compound represented by the formula [2]:

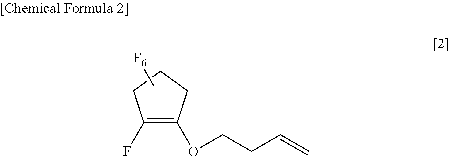

5. A fluorine-containing polymer, characterized by including structural units represented by the formula [3] and/or [4]:

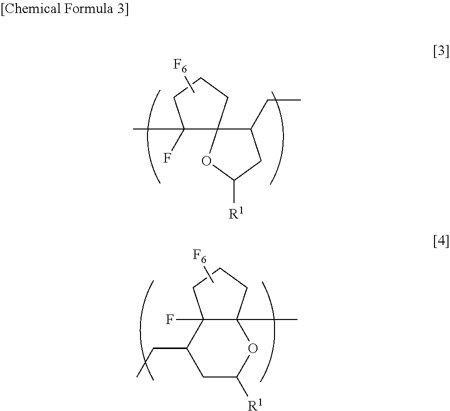

(wherein R¹ represents an alkyl group having 1 to 12 carbon atoms, which may be substituted);
6. The fluorine-containing polymer of 5, wherein R¹ is an alkyl group having 1 to 12 carbon atoms or a fluoroalkyl group having 1 to 12 carbon atoms;
7. The fluorine-containing polymer of any of 4 to 6, wherein a refractive index at a wavelength of 633 nm is at 1.30 to 1.45;
8. A varnish including the fluorine-containing polymer of any of 4 to 7;
9. A thin film including the fluorine-containing polymer of any of 4 to 7;
10. A method for preparing a fluorine-containing polymer including structural units of the formula [3] and/or [4], characterized by polymerizing a 1,6-diene ether compound of 2 or 3, or polymerizing a 1,6-diene ether compound of 2 or 3 and a 1,6-diene ether compound represented by the formula [2], in the presence of a radical generating agent:

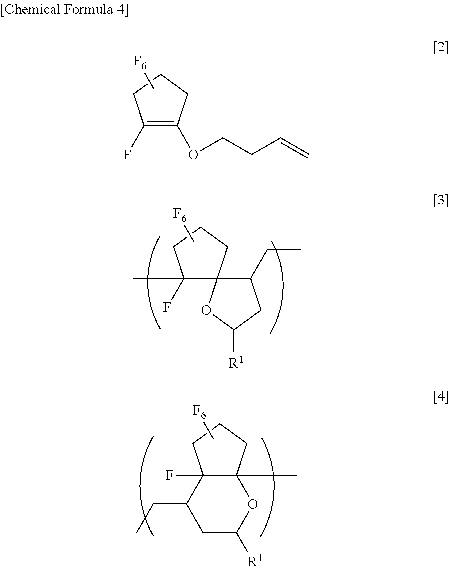

(wherein R¹ represents an alkyl group having 1 to 12 carbon atoms, which may be substituted.)

### Advantageous Effects of the Invention

The fluorine-containing polymer of the invention is a high-functional polymer exhibiting a low refractive index, high glass transition point, high transparency and solvent solubility, and a variety of applications thereof as a coating material and a bulk material will be expected. For instance, the polymer is effective for application in the high-tech fields of optical materials such as a low-reflection film and an optical waveguide clad, and semiconductor materials such as of a pellicle, a resist and the like in semiconductor lithography along further with protective materials, insulating materials, water-repellent materials and the like.

This fluorine-containing polymer can provide materials capable of imparting thereto not only high transparency, high heat resistance, low refractive index, low dielectric constant and low surface energy, but also other desired characteristics by controlling the refractive index and heat resistance.

### EMBODIMENT FOR CARRYING OUT THE INVENTION

The invention is now described in more detail.

The 1,6-diene ether compound of the invention is one represented by the above-indicated formula [1].

In the formula [1], R¹ represents an alkyl group having 1 to 12 carbon atoms or an aryl group having 6 to 18 carbon atoms, which may be substituted.

Specific examples of R¹ include a linear alkyl group, a linear fluoroalkyl group, a branched alkyl group, a branched fluoroalkyl group, a cyclic alkyl group, a cyclic fluoroalkyl group, a phenyl group and the like.

Of these, a linear alkyl group, a linear fluoroalkyl group, a branched alkyl group, a branched fluoroalkyl group, a cyclic alkyl group and a cyclic fluoroalkyl group are preferred from the standpoint of polymerization reactivity of the resulting compound. In view of obtaining polymers having a low refractive index, a linear fluoroalkyl group, a branched fluoroalkyl group and a cyclic fluoroalkyl group are more preferred, and in view of biosafety of the resulting compound, a fluoroalkyl group having 1 to 6 carbon atoms is most preferred.

Typical examples of the linear, branched or cyclic alkyl group include a methyl group, an ethyl group, an n-propyl group, an isopropyl group, a cyclopropyl group, an n-butyl group, an isobutyl group, an s-butyl group, a tert-butyl group, a cyclobutyl group, a 1-methylcyclopropyl group, a 2-methylcyclopropy group, an n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a cyclopentyl group, a 1-methylcyclobutyl group, a 2-methylcyclobutyl group, a 3-methylcyclobutyl group, a 1,2-dimethylcyclopropyl group, a 2,3-dimethylcyclopropyl group, a 1-ethylcyclopropyl group, a 2-ethylcyclopropyl group, an n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, a 1-ethyl-2-methyl-n-propyl group, a cyclohexyl group, a 1-methylcyclopentyl group, a 2-methylcyclopentyl group, a 3-methylcylopentyl group, a 1-ethylcyclobutyl group, a 2-ethylcyclobutyl group, a 3-ethylcyclobutyl group, a 1,2-dimethylcyclobutyl group, a 1,3-dimethylcyclobutyl group, a 2,2-diemthylcyclobutyl group, a 2,3-dimethylcyclobutyl group, a 2,4-dimethylcyclobutyl group, a 3,3-dimethylcyclobutyl group, a 1-n-propylcyclopropyl group, a 2-n-propylcyclopropyl group, a 1-isopropylcyclopropyl group, a 2-isopropylcyclopropyl group, a 1,2,2-trimethylcyclopropyl group, a 1,2,3-trimethylcyclopropyl group, a 2,2,3-trimethylcyclopropyl group, a 1-ethyl-2-methylcyclopropyl group, a 2-ethyl-1-methylcyclopropyl group, a 2-ethyl-2-methylcyclopropyl group, a 2-ethyl-3-methylcyclopropyl group, an n-heptyl group, an n-octyl group, an n-nonyl group, an n-decyl group and the like.

These alkyl groups may be further substituted.

Typical examples of the linear, branched or cyclic fluoroalkyl group include a trifluoromethyl group, a pentafluoroethyl group, a 2,2,2-trifluoroethyl group, a heptafluoropropyl group, a 2,2,3,3,3-pentafluoropropyl group, a 2,2,3,3-tetrafluoropropyl group, a 2,2,2-trifluoro-1-(trifluoromethyl)ethyl group, a nonafluorobutyl group, a 4,4,4-trifluorobutyl group, an undecafluoropentyl group, a 2,2,3,3,4,4,5,5,5-nonafluoropentyl group, a 2,2,3,3,4,4,5,5-octafluoropentyl group, a tridecafluorohexyl group, a 2,2,3,3,4,4,5,5,6,6,6-undecafluorohexyl group, a 2,2,3,3,4,4,5,5,6,6-decafluorohexyl group, a 3,3,4,4,5,5,6,6,6-nonafluorohexyl group and the like.

It will be noted that the 1,6-diene ether compound of the invention should preferably be liquid at 25° C.

The method for preparing the 1,6-diene ether compound of the invention is not limited. As one instance, mention is made of a method including the first step of preparing a homoallyl alcohol derivative, and the second step of preparing a 1,6-diene ether compound from the homoallyl alcohol derivative used as one of starting materials.

The first step of preparing a homoallyl alcohol derivative can be carried out by a method of reaction between an aldehyde and an allyl metal compound, or by a method wherein a carboxylic ester and an allyl metal compound are reacted, followed by reduction in the reaction system.

The method of reaction between an aldehyde and an allyl metal compound includes subjecting the allyl metal compound to nucleophilic addition to the aldehyde to prepare a homoallyl alcohol derivative.

The kind of allyl metal compound used includes an allyl magnesium halide, an allyl aluminum halide, allyl lithium, an allyl trialkyltin, an allyl tin halide, an allyl trialkylsilane, an allyl silyl halide, an allyl trialkoxysilane and the like, of which allyl magnesium bromide is preferred.

The amount of the allyl metal compound is preferably at 0.1 to 10 times by mole, preferably at 0.2 to 5 times by mole, relative to the aldehyde.

The solvents used for the reaction may be any types of solvents so far as they do not influence the reaction. For example, there can be used: aliphatic hydrocarbons such as pentane, hexane, heptane, octane, cyclohexane and the like; ethers such as diethyl ether, diisopropyl ether, dibutyl ether, cyclopentyl methyl ether, tetrahydrofuran, 1,4-dioxane and the like; aromatic hydrocarbons such as benzene, toluene, xylene, mesitylene and the like; and alcohols such as methanol, ethanol, 2-propanol, 2-butanol and the like.

The amount of the solvent is generally at 0.1 to 100 parts by weight per 1 part by weight of aldehyde, preferably at 1 to 20 parts by weight, from the standpoint of safety and economy.

The reaction temperature is generally at −100 to 200° C., preferably at −20 to 30° C.

The reaction time is generally at 0.1 to 48 hours, preferably at 12 to 24 hours.

After completion of the reaction, an ordinary after-treatment is carried out and purification may be made, if necessary, thereby obtaining an intended product.

The purification method includes a method using silica gel column chromatography, a method using distillation and the like, of which the distillation method is preferred in view of the simplicity in operation.

On the other hand, the method of reduction in a reaction system after reaction between a carboxylic ester and an allyl metal compound is one wherein a reaction intermediate formed by subjecting an allyl metal compound to nucleic addition to a carboxylic ester is reduced by addition of a metal reducing agent or a reaction promoter capable of promoting the Meerwein-Ponndorf-Verley reduction, thereby preparing a homoallyl alcohol derivative.

The kind of allyl metal compound used includes an allyl magnesium halide, an allyl aluminum halide, allyl lithium, an allyl trialkyltin, an allyl tin halide, an allyl trialkylsilane, an allyl silyl halide, an allyl trialkoxysilane and the like, of which allyl magnesium bromide is preferred.

The amount of the allyl metal compound is preferably at 0.1 to 10 times by mole, preferably at 0.2 to 5 times by mole, relative to the carboxylic ester.

The kind of metal reducing agent used includes: an aluminum hydride compound such as lithium aluminum hydride, diisobutylaluminum hydride, bis(2-methoxyethyoxy)aluminum sodium hydride or the like; a boron hydride compound such as boron sodium hydride, boron lithium hydride or the like; or an alkali metal hydride such as sodium hydride, potassium hydride or the like. Of these, use of boron sodium hydride is preferred.

The amount of the metal reducing agent is at 0.1 to 10 times by mole, preferably at 0.2 to 5 times by mole, relative to the carboxylic ester.

The kind of reaction promoter used includes an alcohol such as methanol, ethanol, 2-propanol, 2-butanol or the like, of which 2-propanol is preferred.

As a solvent used for the reaction, a variety of solvents may be used in so far as they do not adversely influence the reaction and include: aliphatic hydrocarbons such as pentane, hexane, heptane, octane, cyclohexane and the like; ethers such as diethyl ether, diisopropyl ether, dibutyl ether, cyclopentyl methyl ether, tetrahydrofuran, 1,4-dioxane and the like; and aromatic hydrocarbons such as benzene, toluene, xylene, mesitylene and the like.

The amount of the solvent is at 0.1 to 100 parts by weight relative to 1 part by weight of a carboxylic ester, preferably at 1 to 20 parts by weight in view of safety and economy.

The reaction temperature is generally at −100 to 200° C., preferably at −20 to 100° C.

The reaction time is generally at 0.1 to 48 hours, preferably at 12 to 24 hours.

After completion of the reaction, an ordinary after-treatment is carried out and purification may be made, if necessary, to obtain an intended product.

The purification method includes a method using silica gel column chromatography or a distillation method, of which the distillation method is preferred in view of the simplicity in operation.

The second step is one wherein the homoallyl alcohol derivative obtained in the above first step and OFCP are reacted in the presence of a base to obtain a 1,6-diene ether compound.

According to this reaction, the homoallyl alcohol derivative is converted to a corresponding alkoxide by the action of a base, followed by reaction between the alkoxide and OFCP to obtain a 1,6-diene ether compound.

In this case, the amount of OFCP is preferably at 0.1 to 10 times by mole, preferably at 0.2 to 5 times by mole, relative to the homoallyl alcohol derivative.

Usable bases include: alkali metal hydroxides such as lithium hydroxide, sodium hydroxide, potassium hydroxide and the like; alkali metal alkoxides such as sodium methoxide, sodium ethoxide, sodium tert-butoxide, potassium tert-butoxide and the like; and alkali metal hydrides such as sodium hydride and the like, of which potassium hydroxide is preferred.

The amount of the base is at 0.5 to 10 times by mole, preferably at 1 to 5 times by moles, relative to the homoallyl alcohol derivative substrate.

For the reaction, no solvent may be used. If a solvent is used, no limitation is placed thereon so far as it does not adversely influence the reaction. Examples include: aliphatic hydrocarbons such as pentane, hexane, heptane, octane, cyclohexane and the like; ethers such as diethyl ether, diisopropyl ether, dibutyl ether, cyclopentyl methyl ether, tetrahydrofuran, 1,4-dioxane and the like; and aromatic hydrocarbons such as benzene, toluene, xylene, mesitylene and the like.

Although no limitation is placed on the amount of solvent, too large an amount is unfavorable from the aspect of economy.

The reaction temperature is generally at −100 to 200° C., preferably at −20 to 20° C.

The reaction time is generally at 0.1 to 24 hours, preferably at 1 to 5 hours.

After completion of the reaction, an ordinary after-treatment is carried out and purification may be made, if necessary, to obtain an intended product.

The purification method includes a method using silica gel column chromatography or a distillation method, of which the distillation method is preferred in view of the simplicity in operation.

The fluorine-containing polymer of the invention can be obtained by polymerizing a 1,6-diene ether compound represented by the following formula [1] in the presence of a radical generating agent, or by polymerizing a 1,6-diene ether compound represented by the formula [1] and a 1,6-diene ether compound represented by the formula [2] in the presence of a radical generating agent:

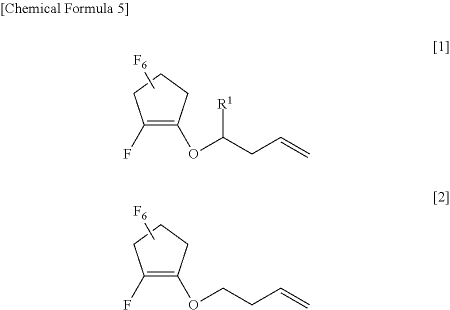

(wherein R¹ represents an alkyl group having 1 to 12 carbon atoms, which may be substituted.)

The compounds represented by the formula [1] may be used singly or in combination of two or more.

More particularly, the fluorine-containing polymer of the invention includes a homopolymer obtained by polymerizing one type of compound represented by the formula [1], a copolymer obtained by polymerizing two or more types of compounds represented by the formula [1], a copolymer obtained by polymerizing one type of compound represented by the formula [1] and a compound represented by the formula [2], and a copolymer obtained by polymerizing two or more types of compounds represented by the formula [1], and a compound represented by the formula [2].

For the method of polymerization in the presence of a radical generating agent, there can be used bulk polymerization, solution polymerization, suspension polymerization, emulsion polymerization and the like. In the practice of the invention, bulk polymerization is preferably used.

The radical generating agent is not limited in type, and examples include: peroxides such as acetyl peroxide, benzoyl peroxide, methyl ethyl ketone peroxide, cyclohexanone peroxide, hydrogen peroxide, tert-butyl hydroperoxide, cumene hydroperoxide, di-tert-butyl peroxide, dicumyl peroxide, dilauroyl peroxide, tert-butyl peroxyacetate, tert-butyl peroxypivarate and the like; azo compounds such as 2,2′-azobisisobutyronitrile, 2,2′-azobis(2,4-dimethylvaleronitrile), (1-phenylethyl)azodiphenylmethane, 2,2′-azobis(4-methoxy-2,4-dimethylvaleronitrile), dimethyl 2,2′-azobisisobutyrate, 2,2′-azobis(2-methylbutyronitrile), 1,1′-azobis(1-cyclohexanecarbonitrile), 2-(carbamoylazo)isobutyronitrile, 2,2′-azobis(2,4,4-trimethylpentane), 2-phenylazo-2,4-dimethyl-4-methoxyvaleronitrile, 2,2′-azobis(2-methylpropane) and the like; and persulfate salts such as ammonium persulfate, sodium persulfate, potassium persulfate and the like.

The polymerization reaction temperature may be appropriately selected depending on the type of radical generating agent used and is preferably at 60 to 120° C.

The polymerization time is preferably at 4 to 48 hours.

According to the polymerization reaction set out above, there can be obtained a fluorine-containing polymer that is assumed to contain structural units represented by the formula [3] and/or [4].

In the fluorine-containing polymer, the content of the structural units represented by the formula [3] and/or [4] should preferably be at 1 to 100 wt % in the polymer.

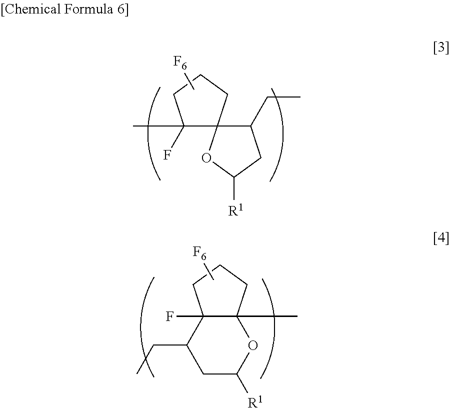

(wherein R¹ represents an alkyl group having 1 to 12 carbon atoms, which may be substituted.)

The fluorine-containing polymer of the invention exhibits such a low refractive index as mentioned hereinabove and preferably has a refractive index of 1.30 to 1.45 at a wavelength of 633 nm.

The fluorine-containing polymer of the invention stated hereinabove can be used as a varnish because of its solvent solubility.

The solvents used for the preparation of varnish are not limited so far as they enable the fluorine-containing polymer and additives added thereto, if required, to be uniformly dissolved or dispersed therein, and those that are able to uniformly dissolve the fluorine-containing polymer are preferred.

Although the solid content in the varnish is not limited so far as it is within a range where the fluorine-containing polymer can be uniformly dissolved or dispersed, the solid content is preferably at 0.1 to 50 wt %, more preferably at 0.1 to 20 wt %.

It will be noted that a variety of additives showing desired characteristics may be added, if necessary, to the varnish of the invention.

As a solvent used for the preparation of varnish, mention is made, for example, of: ester solvents such as diethyl oxalate, ethyl acetoacetate, ethyl acetate, isobutyl acetate, ethyl butyrate, ethyl lactate, ethyl 3-methoxy-propionate, methyl 2-hydroxyisobutyrate and the like; ketone solvents such as methyl ethyl ketone, methyl isobutyl ketone, 2-hexanone, cyclohexanone and the like; propylene glycol solvents such as propylene glycol monomethyl ether, propylene glycol monomethyl ether acetate and the like; cellosolve solvents such as methyl cellosolve, methyl cellosolve acetate and the like; ether solvents such as dibutyl ether, tetrahydrofuran, 1,4-dioxane and the like; alcohol solvents such as ethanol, isopropanol, isopentyl alcohol and the like; aromatic hydrocarbon solvents such as toluene, xylene and the like; and chlorinated hydrocarbon solvents such as dichloromethane, 1,2-dichloroethane, trichloroethylene and the like. These solvents may be used singly or in the form of a mixed solvent of two or more, if necessary.

The above-stated varnish can be coated onto a substrate or the like and heated, if necessary, to form a thin film.

The coating method is arbitrary and mention is made, for example, of a roll coating method, a microgravure coating method, a gravure coating method, a flow coating method, a bar coating method, a spray coating method, a die coating method, a spin coating method, a dip coating method and the like. Among from these methods, the most suitable coating method can be determined while taking the balance of productivity, film thickness controllability, yield and the like into consideration.

It will be noted that the method of preparing a thin film is not limited to the coating methods indicated above, but other techniques such as a vapor deposition method may be used.

### EXAMPLES

The invention is more particularly described by way of Synthetic Examples and Examples, and the invention should not be construed as being limited to the following Examples. It will be noted that analyzing devices and conditions used in the Examples are indicated below.

### [1] ¹H NMR

### (1) Synthetic Examples 1, 2 and Examples 1, 3

Apparatus: GSX-400, made by JEOL Ltd.
Solvent for measurement: CDCl₃
Reference substance: tetramethylsilane (0 ppm)

### (2) Examples 5, 6

Apparatus: JNM-ECX300, made by JEOL Ltd.
Solvent for measurement: CDCl₃ (Example 5),
   (CD₃)₂CO (Example 6)
Reference substance: tetramethylsilane (0 ppm)

### ¹⁹F NMR

### (1) Synthetic Example 1 and Examples 1, 3

Apparatus: R-1200F, made by Hitachi Ltd.
Solvent for measurement: diethyl ether
Reference substance: trifluoroacetic acid (0 ppm)

### (2) Example 5

Apparatus: INOVA-400, made by Varian
   Technologies Japan, Ltd.
Solvent for measurement: CDCl₃
Reference substance: trifluoroacetic acid (0 ppm)

### [3] Gel Permeation Chromatography (GPC)

### (1) Examples 2, 4

Apparatus: LC-2000 Plus series,
   made by JASCO Corporation
Column: PL gel 5μ MIXED-C×2,
   made by Polymer Laboratories Ltd.
Column temperature: 35° C.
Detector: RI
Eluent: THF
Column flow rate: 1.0 ml/minute

### (2) Example 6

Apparatus: HLC-8220GPC,
   made by Tosoh Corporation
Column: SHODEX GPC-8051×2+SHODEX GPC
   KF-G (guard column)
Reference column: SHODEX GPC KF-800RH×2
Column temperature: 40° C.
Detector: RI
Eluent: THF
Column flow rate: 1.0 ml/minute
Reference column flow rate: 0.2 ml/minute

### [4] Refractive Index

Apparatus: High-speed spectroscopic
   ellipsometry M2000-VI, made by
   J. A. Woollam Japan Co., Inc.

### Synthetic Example 1

### Synthesis of 1,1,1-trifluoro-4-penten-2-ol

Under argon atmosphere, 40 ml (40 mmols) of a 1.0 M allyl magnesium bromide/diethyl ether solution was dropped in 5.68 g (40 mmols) of ethyl trifluoroacetate, cooled to 0° C. The reaction solution was stirred at 0° C. for 30 minutes as it is, after which the temperature was raised to 20° C., followed by further stirring for 2 hours. Next, 5.0 ml (65 mmols) of 2-propanol was added to the reaction solution and heated under reflux for 24 hours. Thereafter, 3.0 M hydrochloric acid was added to the reaction solution and washed three times with a saturated saline solution, followed by drying over anhydrous magnesium sulfate. The solvent was distilled off and the resulting crude product was purified by Kugelrohr distillation (at an oven temperature of 140° C. under an atmospheric pressure) to obtain 3.85 g (yield 69%) of 1,1,1-trifluoro-4-penten-2-ol. The results of ¹H NMR and ¹⁹F NMR of the thus obtained product are indicated below.

¹H NMR (400 MHz): δ 2.20 to 2.30 (1H, m), 2.34 to 2.43 (1H, m), 2.49 to 2.55 (1H, m), 3.95 to 4.03 (1H, m), 5.22 to 5.27 (2H, m), 5.78 to 5.89 (1H, m) ppm.

¹⁹F NMR (56.46 MHz): δ −1.83 (3F, s) ppm.

### Synthetic Example 2

### Synthesis of 1-cyclohexyl-3-buten-1-ol

Under argon atmosphere, 20 ml (20 mmols) of a 1.0 M allyl magnesium bromide/diethyl ether solution was dropped in 2.24 g (20 mmols) of cyclohexane carboxyaldehyde cooled to 0° C. The reaction solution was stirred at 0° C. for 30 minutes as it is, after which the temperature was raised to 20° C., followed by further stirring for 24 hours. Thereafter, 3.0 M hydrochloric acid was added to the reaction solution and washed three times with a saturated saline solution, followed by drying over anhydrous magnesium sulfate. The solvent was distilled off and the resulting crude product was purified by Kugelrohr distillation under reduced pressure (at an oven temperature of 160° C. at 4 mmHg) to obtain 2.70 g (yield 88%) of 1-cyclohexyl-3-buten-1-ol. The results of

¹H NMR of the thus obtained product are indicated below.

¹H NMR (400 MHz): δ 0.93 to 21.41 (6H, m), 1.53 to 1.91 (6H, m), 2.07 to 2.22 (1H, m), 2.26 to 2.38 (1H, m), 3.35 to 3.46 (1H, m), 5.10 to 5.13 (1H, m), 5.13 to 5.17 (1H, m), 5.76 to 5.94 (1H, m) ppm.

### Example 1

### Synthesis of 1-(1,1,1-trifluoro-4-penten-2-yloxy)-2,3,3,4,4,5,5-heptafluorocyclopentene

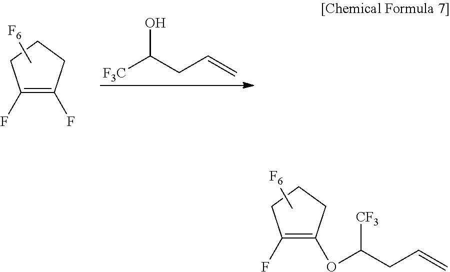

Under argon atmosphere, 0.67 g (12 mmols) of potassium hydroxide was added to 3.39 g (16 mmols) of octafluorocyclopentene (OFCP) and cooled to 0° C., after which 1.12 g (8 mmols) of 1,1,1-trifluoro-4-penten-2-ol obtained in Synthetic Example 1 was dropped in the solution. The reaction solution was stirred at 0° C. for 30 minutes as it is, after which the temperature was raised to 20° C., followed by further stirring for 24 hours. Thereafter, 3.0 M hydrochloric acid was added to the reaction solution and washed three times with a saturated saline solution. Unreacted OFPC was distilled off from the resulting organic phase, followed by drying over anhydrous magnesium sulfate. The resulting crude product was purified by Kugelrohr distillation under reduced pressure (at an oven temperature of 140° C. at 4 mmHg) to obtain 1.55 g (yield 58%) of 1-(1,1,1-trifluoro-4-penten-2-yloxy)-2,3,3,4,4,5,5-heptafluorocyclopentene. The results of ¹H NMR and ¹⁹F NMR of the thus obtained product are indicated below.

¹H NMR (400 MHz): δ 2.65 (2H, dd, J=6 Hz, 6 Hz), 4.76 to 4.86 (1H, m), 5.26 to 5.33 (2H, m), 5.71 to 5.84 (2H, m) ppm.

¹⁹F NMR (56.46 MHz): δ −77.5 to −80.9 (1F, m), −53.2 to −55.0 (2F, m), −37.4 to −40.9 (4F, m), −0.81 (3F, s) ppm.

### Example 2

### Single bulk polymerization of 1-(1,1,1-trifluoro-4-penten-2-yloxy)-2,3,3,4,4,5,5-heptafluorocyclopentene

0.33 g (1.0 mmol) of 1-(1,1,1-trifluoro-4-penten-2-yloxy)-2,3,3,4,4,5,5-heptafluorocyclopentene obtained in Example 1 and 2 mg of benzoyl peroxide (made by Kishida Chemical Co., Ltd., with a water content of 25%)(0.5 mol %) were placed in a glass polymerization tube, followed by repeating three times (1) degassing under cooling to −78° C. and (2) melting at room temperature, and sealing the tube. After polymerization at 80° C. for 24 hours, the resulting polymer was dissolved in a small amount of tetrahydrofuran and dropped in methanol, followed by re-precipitation and decantation. Thereafter, the solvent contained was distilled off under reduced pressure to obtain 0.071 g (yield 22%) of the polymer of 1-(1,1,1-trifluoro-4-penten-2-yloxy)-2,3,3,4,4,5,5-heptafluorocyclopentene. The weight average molecular weight Mw of the polymer, which was measured by GPC in terms of polystyrene conversion, was at 15,800.

### Example 3

### Synthesis of 1-(1-cyclohexyl-3-buten-1-yloxy)-2,3,3,4,4,5,5-heptafluorocyclopentene

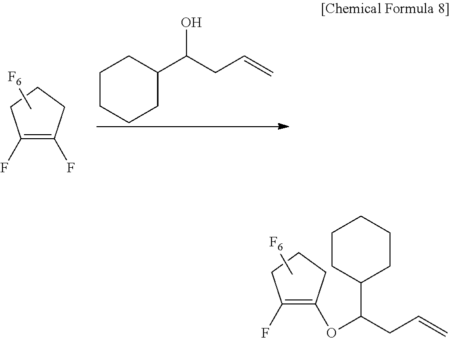

Under argon atmosphere, 0.84 g (15 mmols) of potassium hydroxide was added to 4.24 g (20 mmols) of octafluorocyclopentene (OFCP) and cooled to 0° C., after which 1.10 g (7.7 mmols) of 1-cyclohexyl-3-buten-1-ol obtained in Synthetic Example 2 was dropped in the reaction solution. The reaction solution was stirred at 0° C. for 30 minutes as it is, after which the temperature was raised to 20° C., followed by further stirring for 24 hours. Thereafter, 3.0 M hydrochloric acid was added to the reaction solution and washed three times with a saturated saline solution. Unreacted OFPC was distilled off from the resulting organic phase, followed by drying over anhydrous magnesium sulfate. The resulting crude product was purified by Kugelrohr distillation under reduced pressure (at an oven temperature of 160° C. at 4 mmHg), followed by further purification with silica gel column chromatography (developing solvent: n-hexane) to obtain 1.69 g (yield 64%) of 1-(1-cyclohexyl-3-buten-1-yloxy)-2,3,3,4,4,5,5-heptafluoro-cyclopentene. The results of ¹H NMR and ¹⁹F NMR of the thus obtained product are indicated below.

¹H NMR (400 MHz): δ 0.93 to 1.39 (6H, m), 1.56 to 1.88 (6H, m), 1.95 to 2.48 (2H, m), 3.35 to 3.46 (1H, m), 5.10 to 5.13 (1H, m), 5.13 to 5.17 (1H, m), 5.76 to 5.94 (1H, m) ppm.

¹⁹F NMR (56.46 MHz): δ −81.8 to −87.0 (1F, m), −51.0 to −53.7 (2F, m), −37.2 to −40.0 (2F, m), −35.4 to −37.2 (2F, m) ppm.

### Example 4

### Single bulk polymerization of 1-(1-cyclohexyl-3-buten-1-yloxy)-2,3,3,4,4,5,5-heptafluorocyclopentene

0.35 g (1.0 mmol) of 1-(1-cyclohexyl-3-buten-1-yloxy)-2,3,3,4,4,5,5-heptafluorocyclopentene obtained in Example 3 and 2 mg of benzoyl peroxide (made by Kishida Chemical Co., Ltd., with a water content of 25%) (0.5 mol %) were placed in a glass polymerization tube, followed by repeating three times (1) degassing under cooling to −78° C. and (2) melting at room temperature, and sealing the tube. After polymerization at 80° C. for 24 hours, the resulting polymer was dissolved in a small amount of tetrahydrofuran and dropped in methanol, followed by re-precipitation and decantation. Thereafter, the solvent contained was distilled off under reduced pressure to obtain 0.048 g (yield 14%) of the polymer of 1-(1-cyclohexyl-3-buten-1-yloxy)-2,3,3,4,4,5,5-heptafluoro-cyclopentene. The weight average molecular weight Mw of the polymer, which was measured by GPC in terms of polystyrene conversion, was at 11,600.

### Example 5

### Synthesis of 1-(4-penten-2-yloxy)-2,3,3,4,4,5,5-heptafluorocyclopentane

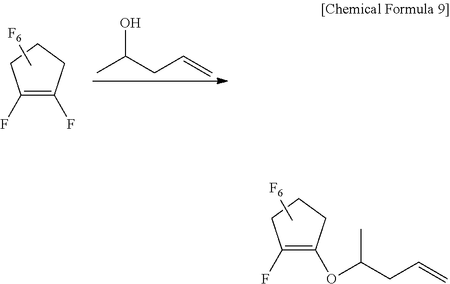

Under argon atmosphere, 1.39 g (25 mmols) of potassium hydroxide was added to 6.36 g (30 mmols) of octafluorocyclopentene (OFCP) and cooled to 0° C., after which 1.72 g (20 mmols) of 4-penten-2-ol was dropped in the reaction solution. The reaction solution was stirred at 0° C. for 10 minutes as it is, after which the temperature was raised to 20° C., followed by further stirring for 24 hours. Thereafter, 3.0 M hydrochloric acid was added to the reaction solution and washed three times with a saturated saline solution. Unreacted OFPC was distilled off from the resulting organic phase, followed by drying over anhydrous magnesium sulfate. The resulting crude product was purified by reduced pressure distillation (at a bath temperature of 180° C. at 4 mmHg) to obtain 5.03 g (yield 91%) of 1-(4-penten-2-yloxy)-2,3,3,4,4,5,5-heptafluorocyclopentene. The results of ¹H NMR and ¹⁹F NMR of the thus obtained product are indicated below.

¹H NMR (300 MHz): δ 1.35 to 1.42 (3H, m), 2.30 to 2.56 (2H, m), 4.69 (1H, dq, J=2 Hz, 6 Hz), 5.09 to 5.22 (2H, m), 5.66 to 5.85 (1H, m) ppm.

¹⁹F NMR (376 MHz): δ −85.4 to −85.7 (1F, m), −53.9 to −54.1 (2F, m), −40.6 to −40.9 (2F, m), −38.8 to −39.0 (2F, m) ppm.

### Example 6

### Single bulk polymerization of 1-(4-penten-2-yloxy)-2,3,3,4,4,5,5-heptafluorocyclopentene

0.83 g (3.0 mmols) of 1-(4-penten-2-yloxy)-2,3,3,4,4,5,5-heptafluorocyclopentene obtained in Example 5 and 4 mg of benzoyl peroxide (made by Kishida Chemical Co., Ltd., with a water content of 25%)(0.5 mol %) were placed in a glass polymerization tube, followed by degassing at room temperature (about 20° C.) at 4 mmHg for 30 minute and sealing the tube. After polymerization at 80° C. for 24 hours, the resulting polymer was dissolved in a small amount of ethyl acetate and dropped in methanol, followed by re-precipitation and decantation. Thereafter, the solvent contained was distilled off under reduced pressure to obtain 0.65 g (yield 78%) of the polymer of 1-(4-penten-2-yloxy)-2,3,3,4,4,5,5-heptafluorocyclopentene. The weight average molecular weight Mw of the polymer, which was measured by GPC in terms of polystyrene conversion, was at 36,700. The results of ¹H NMR of the intended product are indicated below.

¹H NMR (300 MHz): δ 1.15 to 1.69 (3H, m), 1.69 to 3.48 (5H, m), 4.19 to 4.79 (1H, m) ppm.

### Example 7

### Measurement of Refractive Index

Three parts by weight of the polymers obtained in Examples 2, 4 and 6 were, respectively, dissolved in 97 parts by weight of ethyl acetate to prepare varnishes having a solid concentration of 3 wt %. The respective varnishes were coated onto a glass substrate by a spin coating method (300 rpm×5 seconds and subsequently 1,500 rpm×30 seconds). This glass substrate was heated on a hot plate at 60° C. for 30 minutes to remove the solvent from the wet film thereby obtaining films of the polymers obtained in Examples 2, 4 and 6.

The refractive indices of the respective films at a wavelength of 633 nm were measured and found to be at 1.36 (the film of the polymer of Example 2), 1.42 (the film of the polymer of Example 4) and 1.39 (the film of the polymer of Example 6).

1. A 1,6-diene ether compound, characterized by being represented by the formula [1]: 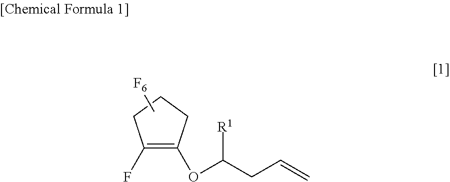
(wherein R¹ represents an alkyl group having 1 to 12 carbon atoms or an aryl group having 6 to 18 carbon atoms, which may be substituted.)

2. The 1,6-diene ether compound as defined in claim 1, wherein R¹ is an alkyl group having 1 to 12 carbon atoms, which may be substituted.

3. The 1,6-diene ether compound as defined in claim 2, wherein R¹ is an alkyl group having 1 to 12 carbon atoms or a fluoroalkyl group having 1 to 12 carbon atoms.

4. A fluorine-containing polymer, characterized by being obtained by polymerizing a 1,6-diene ether compound as defined in claim 3, or polymerizing a 1,6-diene ether compound as defined in claim 3 above and a 1,6-diene ether compound represented by the formula [2]:

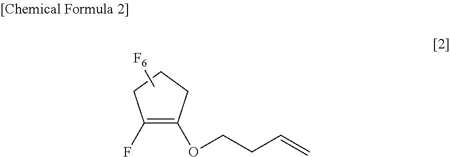

5. A fluorine-containing polymer, characterized by comprising structural units represented by the formula [3] and/or [4]: 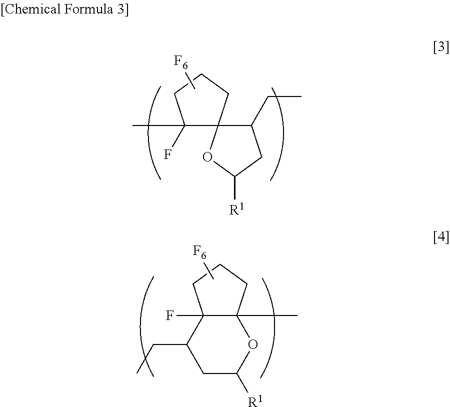
(wherein R¹ represents an alkyl group having 1 to 12 carbon atoms, which may be substituted.)

6. The fluorine-containing polymer as defined in claim 5, wherein R¹ is an alkyl group having 1 to 12 carbon atoms or a fluoroalkyl group having 1 to 12 carbon atoms.

7. The fluorine-containing polymer as defined in claim 6, wherein a refractive index at a wavelength of 633 nm is at 1.30 to 1.45.

8. A varnish comprising the fluorine-containing polymer as defined in claim 7.

9. A thin film comprising the fluorine-containing polymer as defined in claim 7.

10. A method for preparing a fluorine-containing polymer comprising structural units of the formula [3] and/or [4], characterized by polymerizing a 1,6-diene ether compound defined in claim 3, or polymerizing a 1,6-diene ether compound of claim 3 and a 1,6-diene ether compound represented by the formula [2], in the presence of a radical generating agent: 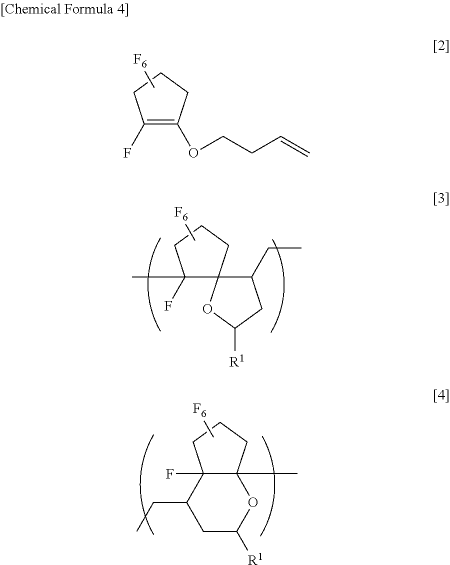
(wherein R¹ represents an alkyl group having 1 to 12 carbon atoms, which may be substituted.)
